(19) United States
(12) Patent Application Publication
(10) Pub. No.: US 2007/0227884 A1
(43) Pub. Date: Oct. 4, 2007

(54) Uricase enzyme biosensors and fabrication method thereof, sensing systems and sensing circuits comprising the same

(75) Inventors: Jung-Chuan Chou, Douliou City (TW); Wei-Li Liao, Taichung City (TW); Yu-Sheng Chen, Kaohsiung City (TW)

Correspondence Address: QUINTERO LAW OFFICE, PC, 2210 MAIN STREET, SUITE 200, SANTA MONICA, CA, 90405, US

(73) Assignee: NATIONAL YUNLIN UNIVERSITY OF SCIENCE AND TECHNOLOGY, YUNLIN (TW)

(21) Appl. No.: 11/448,477

(22) Filed: Jun. 7, 2006

(30) Foreign Application Priority Data
Apr. 4, 2006 (TW) TW095111943

(51) Int. Cl. G01N 33/487

(52) U.S. Cl. 204/403.01

(57) ABSTRACT

A uricase enzyme biosensor and fabrication method thereof. The uricase enzyme biosensor includes a metal oxide semiconductor field effect transistor, a sensing unit including a substrate, a titanium dioxide film formed thereon and a uricase enzyme sensing film formed on the titanium dioxide film, and a conductive wire connecting with the metal oxide semiconductor field effect transistor and the sensing unit. The invention also provides a sensing system and a sensing circuit including the biosensor.

### BACKGROUND OF THE INVENTION

1. Field of the Invention

The invention relates to a biosensor, and in particular to a uricase enzyme biosensor, a sensing system and a sensing circuit comprising the biosensor.

2. Description of the Related Art

Unusual uric acid values are symptoms of many illnesses such as gout, hyperuricemia and so on. Hence, the uric acid values in blood or urine are important indexes of human health, particularly for liver and kidney function. Conventional organic quantitative analytical methods used to analyze uric acid values have many drawbacks such as complicated operation, long analysis time, high cost, unsuitability for detection in a large number of samples and sequential detection processes. Thus, the development of a simple uricase enzyme biosensor to detect the uric acid concentration in blood to assist in medical diagnosis and daily health care is desirable.

In 1970, P. Bergveld (ref.[1], P. Bergveld, “Development of an Ion-sensitive Solid-State Device for Neurophysiological Measurements”, IEEE Transactions on Biomedical Engineering, Vol. Bio-Med. Eng. 17, pp. 70-71, 1970) presented an ion sensitive field effect transistor (ISFET), in which the original metallic gate of metal-oxide-semiconductor field effect transistor (MOSFET) was replaced with a sensing film. The sensing film was immersed in electrolyte and reacted to produce various interface potentials therebetween, altering the channel current of the device. The pH value of the test solution can thereby be detected.

Besides, J. V. D Spiegel and so on (ref.[2], J. Van der Spiegel, I. Lauks, P. Chan D. Babic, “The Extended Gate Chemical Sensitive Field Effect Transistor as Multi-Species Microprobe”, Sensors and Actuators B, Vol. 4, pp. 291-298, 1983.) presented an extended gate field effect transistor (EGFET) structure, in which the sensing film was disposed on the signal terminal extended from the gate of MOSFET. The MOSFET can be kept away from the chemical environment of the test solution by the extended sensing film.

A number of patents or measurement methods related to the biosensors have been disclosed as summarized hereinafter.

U.S. Pat. No. 6,547,954 (Ikeda, Pub. Date Apr. 15, 2003) described an electrochemical biosensor for quantitating various biochemical substrates in sample such as blood, juice and the like, with the characteristics of accuracy, speed and ease. The biochemical substrates may comprise glucose, cholesterol, lactic acid, uric acid or sucrose.

U.S. Pat. No. 6,753,159 (Lee, Jin Po, Pub. Date Jun. 22, 2004) provided an enzyme-based device and a fabrication method thereof in normal condition. The device comprised a dry phase test strip for detecting uric acid and its concentration in sample (such as urine) and a stabilized uricase-containing working solution. It also provided a fabrication method of a device for maintaining the stability of the working solution, especially for the enzyme components thereof. A one-step uric acid measurement method was also provided.

U.S. Pat. No. 5,837,446 (Stephen N. Cozzette, Graham Davis, Jeanne Itak, Imants R. Lauks, Sylvia Piznik, Nicolaas Smit, Susan Steiner, Paul Van Der Werf, Henry J. Wieck, Randall M., Pub. Date Nov. 17, 1998) described a method of detecting analyte and quantity thereof in sample. The sample contained at least one analyte such as potassium ion, sodium ion, calcium ion, protein, hydrogen peroxide, glucose or uric acid.

U.S. Pat. No. 6,867,059 (Jung Chuan Chou, Yii Fang Wang, Pub. Date Oct. 31, 2002) described an ion sensitive field effect transistor with a hydrogenated amorphous silicon sensing film for measuring temperature parameters in test solution and a measurement method thereof. The pH value and ion concentration of the test solution were also measured by source/drain current and gate voltage.

U.S. Pat. No. 4,927,516 (Shuichiro Yamaguchi, Takeshi Shimomura, Pub. Date May 22, 1990) described a separated type enzyme biosensor with an enzyme film immobilized on the separated structure. The measurement procedure was performed by potentiometer and galvanometer.

U.S. Pat. No. 4,877,582 (Oda and so on, Pub. Date Oct. 31, 1989) described a chemical sensor having a field effect transistor as an electronic transducer for analyzing constituents in liquid. The chemical sensor can prevent external light from reaching the field effect transistor.

U.S. Pat. No. 5,309,085 (Byung Ki Sohn, Pub. Date May 3, 1994) described a measuring circuit with a biosensor utilizing ion sensitive field effect transistors integrated into one chip. The measuring circuit comprised two ion sensitive FET input devices composed of an enzyme FET having an enzyme sensitive membrane on the gate, a reference FET, and a differential amplifier for amplifying the outputs of the enzyme FET and the reference FET.

U.S. Pat. No. 6,897,081 (S. K. Hsiung, Jung-Chuan Chou, Tai-Ping Sun, Wen-Yaw Chung, Yuan-Lung Chin, Chung-We Pan, Pub. Date Apr. 22, 2004) described a device including multi-sensors integrated in a monolithic chip that can simultaneously detect pH, temperature, and photo-intensity, and a detection method thereof. Hsiung also provided a readout circuit. The readout circuit switched on the multiple sensors to read pH, temperature, and photo-intensity in order within a period, reducing chip area and cost. The frame was built by standard 0.5 μm CMOS processes and integrated in a monolithic chip. The extended gate field effect transistor (EGFET) provided compensation of temperature and light to achieve accurate detection results.

U.S. Pat. No. 6,218,208 (Jung Chuan Chou, Wen Yaw Chung, Shen Kanr Hsiung, Tai Ping Sun, Hung Kwei Liao, Pub. Date Apr. 17, 2001) described a multi-layer ion sensor fabricated by thermal evaporation and RF sputtering. The multi-layer sensor comprised SnO₂/SiO₂ gate or SnO₂/Si₃N₄/SiO₂. The sensor had a sensitivity of 56˜58 mV/pH at pH 2˜12, with the advantages of low light damage, simple fabrication, low cost, mass productability and disposability.

### BRIEF SUMMARY OF THE INVENTION

The invention provides a uricase enzyme biosensor comprising a metal oxide semiconductor field effect transistor, a sensing unit comprising a substrate, a titanium dioxide film formed thereon and a uricase enzyme sensing film formed on the titanium dioxide film, and a conductive wire connected with the metal oxide semiconductor field effect transistor and the sensing unit.

The invention provides a method of fabricating a uricase enzyme biosensor comprising providing a metal oxide semiconductor field effect transistor, providing a sensing unit comprising a substrate, a titanium dioxide film formed thereon and a uricase enzyme sensing film formed on the titanium dioxide film, and providing a conductive wire to connect the metal oxide semiconductor field effect transistor and the sensing unit.

The invention also provides a uricase enzyme sensing system comprising the disclosed uricase enzyme biosensor, a reference electrode applying a stabilized voltage, a semiconductor characteristic instrument disposed on the uricase enzyme biosensor and connected with the reference electrode by a conductive wire, and a light-isolation container containing the sensing unit of the uricase enzyme biosensor, the reference electrode and a test solution.

The invention further provides a sensing circuit comprising the disclosed uricase enzyme biosensor and a first and second operational amplifiers comprising an output port, a negative-phase input port and a non-negative-phase input port, wherein the output port and the negative-phase input port of the first operational amplifier are connected to the uricase enzyme biosensor and the non-negative-phase input port thereof is connected to a first current source and a first port of a resistance, and the output port and the negative-phase input port of the second operational amplifier are connected to a second port of the resistance and the non-negative-phase input port thereof is connected to a second current source and the uricase enzyme biosensor.

A detailed description is given in the following embodiments with reference to the accompanying drawings.

### BRIEF DESCRIPTION OF THE DRAWINGS

The invention can be more fully understood by reading the subsequent detailed description and examples with references made to the accompanying drawing, wherein:

FIG. 1 shows a uricase enzyme biosensor of the invention.

FIG. 2 shows a uricase enzyme sensing system of the invention.

FIG. 3 shows a uricase enzyme sensing circuit of the invention.

FIG. 4 shows a relationship between various Ar/O₂ flow ratios and pH sensitivity.

FIG. 5A shows a relationship between response voltage and time of a uricase enzyme biosensor of the invention.

FIG. 5B shows a sensitivity curve of a uricase enzyme biosensor of the invention.

FIG. 6 shows a relationship between output voltage (Vₒᵤₜ) and substrate voltage (V_{B}) of a uricase enzyme biosensor of the invention.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

The invention provides a uricase enzyme biosensor comprising a metal oxide semiconductor field effect transistor, a sensing unit comprising a substrate, a titanium dioxide film formed thereon and a uricase enzyme sensing film formed on the titanium dioxide film, and a conductive wire connected with the metal oxide semiconductor field effect transistor and the sensing unit.

The metal oxide semiconductor field effect transistor (MOSFET) can keep away from a test solution due to the uricase enzyme sensing film extended from the gate of the MOSFET, thus reducing instability of the semiconductor device and avoiding signal interference in the solution.

The substrate of the sensing unit may be a semiconductor substrate such as a p-type semiconductor substrate, with a crystal face of (100), suitable for the deposition of TiO2 film. The conductive wire connected with the metal oxide semiconductor field effect transistor and the sensing unit may be an aluminum wire.

The sensing unit may be covered by an insulating layer such as epoxy, exposing the uricase enzyme sensing film.

Titanium dioxide film provides a high refractive index, high dielectric constant, high hardness, high chemical stability, optimal insulating properties and wear-resistance. The rutile and ilmenite structures belonging to titanium dioxide have an optical band gap of 3.05 eV and 3.24 eV, respectively. If the rutile and ilmenite structures are irradiated by different light sources having a wavelength of less than 410 nm and 385 nm, respectively, electrons in valance bands can be excited to conduction bands. Additionally, an anti-corrosion titanium dioxide can endure various solutions with extreme pH values.

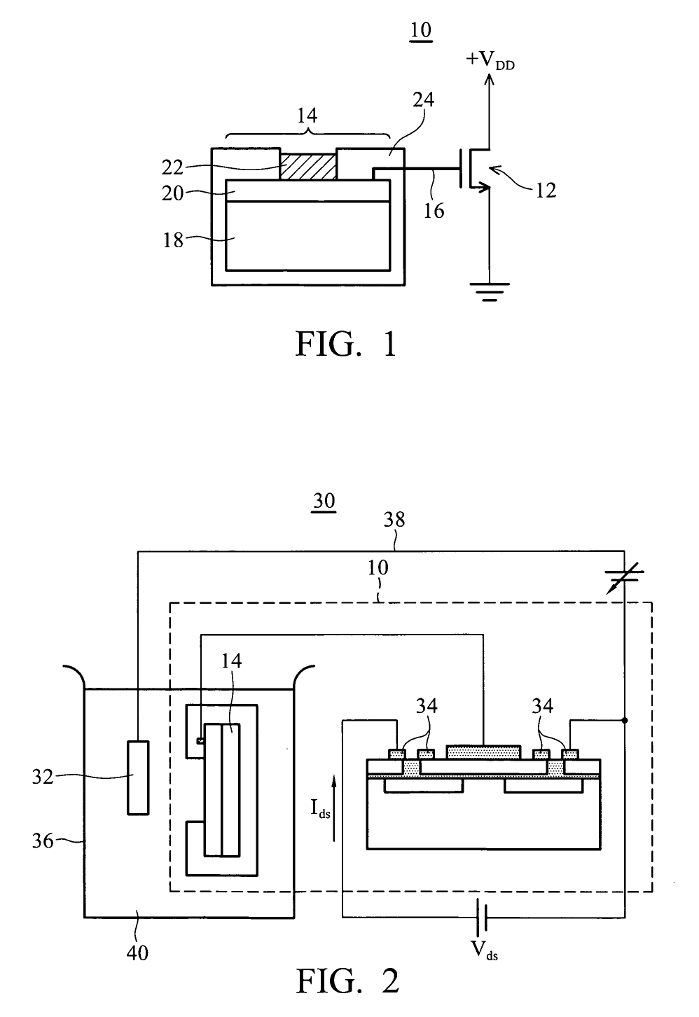

The uricase enzyme biosensor of the invention is disclosed in FIG. 1. A uricase enzyme biosensor 10 comprises a metal oxide semiconductor field effect transistor 12 and a sensing unit 14 connected therewith by a conductive wire 16. The sensing unit 14 comprises a substrate 18, a titanium dioxide film 20 formed thereon and a uricase enzyme sensing film 22 formed on the titanium dioxide film 20. The sensing unit 14 is further covered by an insulating layer 24, exposing the uricase enzyme sensing film 22 contacted with a test solution.

The invention provides a method of fabricating a uricase enzyme biosensor, comprising the following steps. A metal oxide semiconductor field effect transistor is provided. A sensing unit comprising a substrate, a titanium dioxide film formed thereon and a uricase enzyme sensing film formed on the titanium dioxide film is then provided. A conductive wire is provided to connect the metal oxide semiconductor field effect transistor and the sensing unit.

The substrate of the sensing unit may be a semiconductor substrate such as a p-type semiconductor substrate, with a crystal face of (100), suitable for the deposition of TiO2 film. The conductive wire connected with the metal oxide semiconductor field effect transistor and the sensing unit may be an aluminum wire.

The titanium dioxide film can be formed by sputtering such as radio frequency (RF) sputtering, with a working pressure of about 10˜40 mTorr, preferably 30 mTorr, a sputtering duration of about 0.5˜1.5 hour, preferably 1 hour, and a RF power of about 120˜180 W, preferably 150 W. The sputtering may utilize reaction gases such as argon and oxygen, with a flow ratio of about 1:1˜4:1.

RF sputtering is the most popular method for growing titanium dioxide films. The method can sputter insulating materials or high-activity metals and then a large-area and uniform film can be obtained. Additionally, RF sputtering can be performed under lower pressure.

The uricase enzyme sensing film is formed on the titanium dioxide film by a method such as gel entrapment, comprising the following steps. A light-sensitive polymer and a urate oxidase are mixed in a phosphate buffer solution. Next, the solution is titrated on the titanium dioxide film. The solution is then photopolymerized to form a uricase enzyme sensing film immobilized on the titanium dioxide film. The light-sensitive polymer may comprise polyvinyl alcohol. The light-sensitive polymer and the urate oxidase solution may have a weight ratio of about 5:1˜30:1. The photopolymerizing may occur by exposure of UV light to form the uricase enzyme sensing film which urate oxidase is entrapped by the light-sensitive polymer gel.

During the biosensor fabrication, the surface of the sensing unit is further covered by an insulating layer such as epoxy, exposing the uricase enzyme sensing film.

The invention also provides a uricase enzyme sensing system comprising the disclosed uricase enzyme biosensor, a reference electrode applying a stabilized voltage, a semiconductor characteristic instrument disposed on the uricase enzyme biosensor and connected with the reference electrode by a conductive wire, and a light-isolation container containing the sensing unit of the uricase enzyme biosensor, the reference electrode and a test solution.

The reference electrode may be an Ag/AgCl reference electrode. The semiconductor characteristic instrument may be a current/voltage instrument such as Keithley 236 for measuring characteristics such as drain current and gate voltage and further processing the data of electric signals. The conductive wire connected with the semiconductor characteristic instrument and the reference electrode may be an aluminum wire. To avoid light-sensitive affection, the light-isolation container may be a dark box. The test solution may comprise uric acid-containing solutions with different concentrations.

The sensing system further comprises a temperature controller for controlling the temperature of the sensing unit comprising a temperature control center, a thermocouple and a heater. The thermocouple and the heater are connected to the temperature control center, respectively.

After a test solution is poured into the light-isolation container, the uricase enzyme sensing unit, reference electrode and thermocouple are immersed into the solution to measure the uric acid concentration of the test solution. The temperature of the solution is adjusted by the heater. The data measured by the uricase enzyme sensing unit and the reference electrode is then transmitted to the semiconductor characteristic instrument to readout the drain current and gate voltage values. Finally, the uric acid concentration is determined by the readout values.

Further, the uric acid of the test solution will react with urate oxidase. The reaction processes therebetween are illustrated in following formulas (1.1) and (1.2)

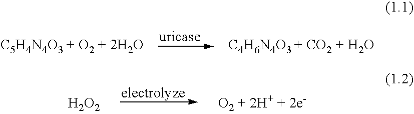

As formula (1.1), uric acid (C₅H₄N₄O₃) is decomposed into allantoin (C₄H₆N₄O₃) and hydrogen peroxide (H₂O₂) by catalysis of urate oxidase. The hydrogen peroxide (H₂O₂) is then electrolyzed to produce hydrogen ions (H⁺) and electrons (e⁻) by the reference electrode. The interface potential between the uricase enzyme sensing film and solution alters as the hydrogen ion concentration alters. The voltage data is then transmitted to the instrument amplifier by the conductive wire to amplify the signals and recorded in personal computer (PC). The output voltage increases with increased uric acid concentration.

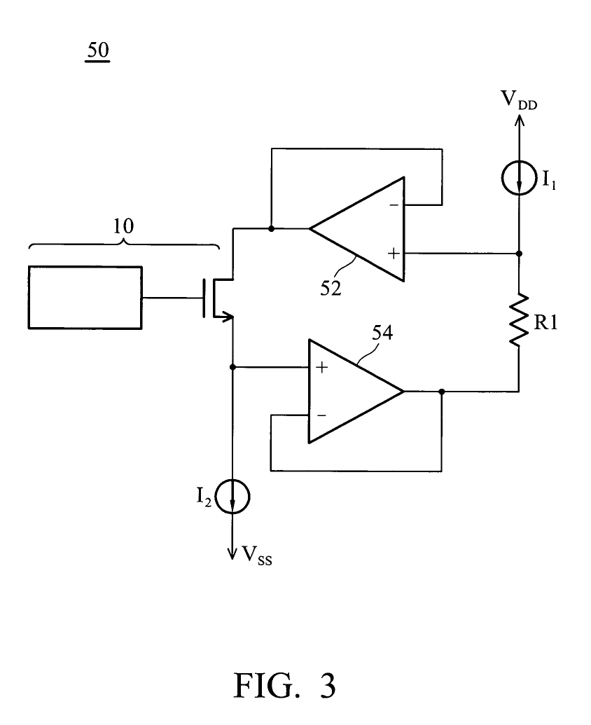

The uricase enzyme sensing system of the invention is disclosed in FIG. 2. The uricase enzyme sensing system 30 comprises the disclosed uricase enzyme biosensor 10, a reference electrode 32, a semiconductor characteristic instrument 34 disposed on the uricase enzyme biosensor 10 and connected with the reference electrode 32 by a conductive wire 38, and a light-isolation container 36 containing the sensing unit 14 of the uricase enzyme biosensor 10, the reference electrode 32 and a test solution 40.

The invention further provides a sensing circuit comprising the disclosed uricase enzyme biosensor, a first operational amplifier comprising an output port, a negative-phase input port and a non-negative-phase input port, and a second operational amplifier comprising an output port, a negative-phase input port and a non-negative-phase, input port. The output port and the negative-phase input port of the first operational amplifier are connected to the uricase enzyme biosensor and the non-negative-phase input port thereof is connected to a first current source and a first port of a resistance. The output port and the negative-phase input port of the second operational amplifier are connected to a second port of the resistance and the non-negative-phase input port thereof is connected to a second current source and the uricase enzyme biosensor.

The first and second operational amplifiers acted as negative feedback voltage buffers exhibit two-stage operational amplification.

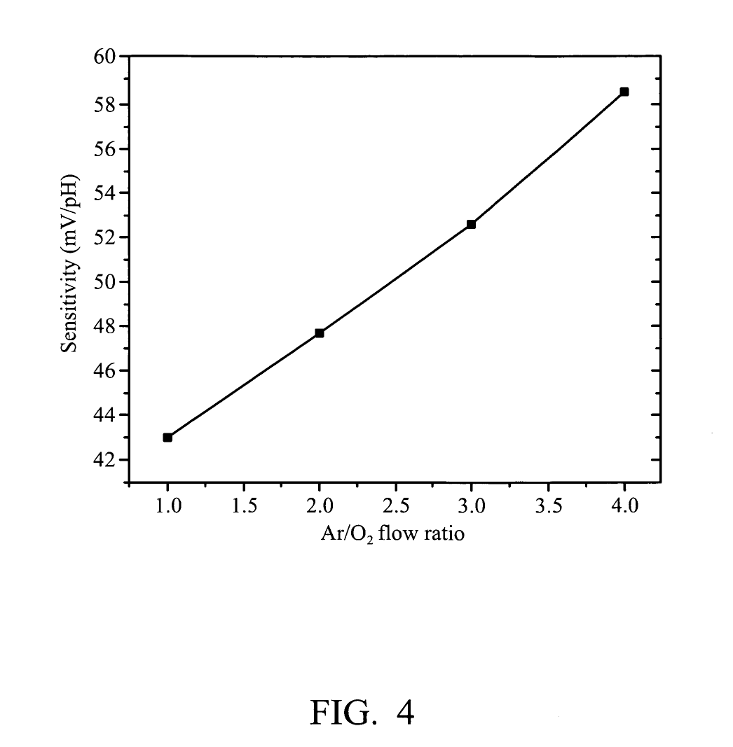

The uricase enzyme sensing circuit of the invention is disclosed in FIG. 3. The sensing circuit 50 comprises the disclosed uricase enzyme biosensor 10, a first operational amplifier 52 comprising an output port, a negative-phase input port and a non-negative-phase input port, and a second operational amplifier 54 comprising an output port, a negative-phase input port and a non-negative-phase input port. The output port and the negative-phase input port of the first operational amplifier 52 are connected to the uricase enzyme biosensor 10 and the non-negative-phase input port thereof is connected to a first current source I₁ and a first port of a resistance R1. The output port and the negative-phase input port of the second operational amplifier 54 are connected to a second port of the resistance R1 and the non-negative-phase input port thereof is connected to a second current source I₂ and the uricase enzyme biosensor 10.

The voltage drop (V_{DS}) between the drain and source of the MOSFET is set to a working point. We suppose the operational amplifier (OPA) is ideal (the gain is infinite and it has visual ground characteristic). The V_{DS} of the MOSFET and the voltage drop produced from that I₁ flowing through R₁ can be equal due to the disposition of the negative feedback voltage buffers composed of the OPA₁ and OPA₂. Thus, V_{DS} can be adjusted by altering I₁ and R₁.

Examples (the response voltage changes with increasing pH value of the solution)

Uricase Enzyme Sensing Unit Preparation

1. Titanium Dioxide Film Preparation

A p-type silicon substrate was provided. A standard cleaning procedure was performed to remove impurities such as particles or silicon oxide on the surface of the wafer to improve subsequently formed film quality. A titanium dioxide film was then sputtered on the silicon substrate by RF sputtering utilizing a titanium target having a 2 inch diameter with 99.99% purity. The sputtering parameters are cited in Table 1.

TABLE 1

| Parameters | Conditions |
|---|---|
| Substrate temperature(° C.) | 25 |
| Gas pressure (mTorr) | 30 |
| Gas flow ratio (Ar/O₂) | 80 sccm/20 sccm |
| RF power (W) | 150 |
| Sputtering duration (Hour) | 1 |
| Annealing temperature (° C.) | 700 |
| Annealing time (Hour) | 1 |
| Annealing gas | O₂ |

Argon and other reaction gases (such as high-purity oxygen) were provided by DRY ICE. Before sputtering, the pressure of the chamber was reduced to about 5 mTorr by a rotary pump and then continuously reduced to less than 3×10⁻⁶ Torr by a turbo pump. The flow rates of argon and oxygen can be controlled by a mass flow controller (MFC).

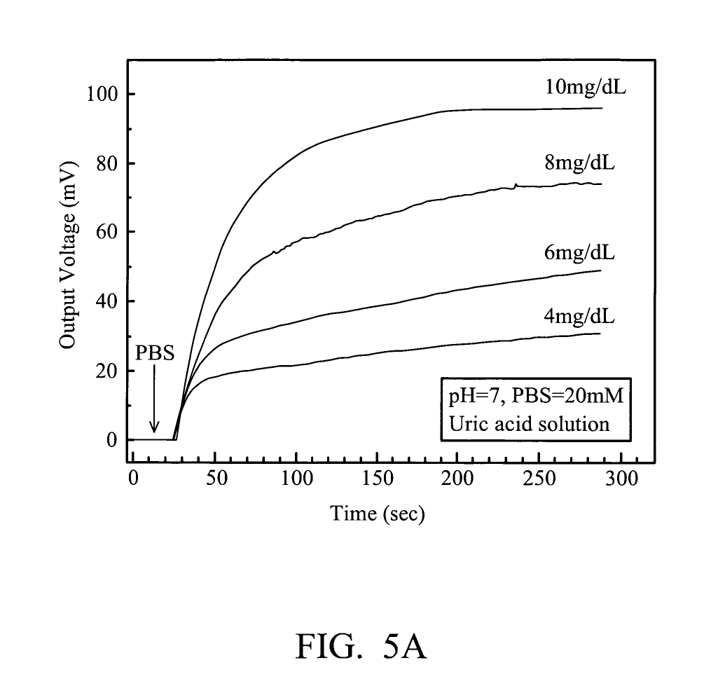

The titanium dioxide films of the invention were prepared by conducting various Ar/O₂ flow ratios. The sensitivity of the titanium dioxide film was analyzed in pH 1˜13 solutions. The Ar/O₂ flow ratios were set to 4/1, 3/1, 2/1 and 1/1. The RF power was 150 W. The sputtering pressure was 30 mTorr and the sputtering duration was 1 hour. The results indicated that when the gas flow ratio was reduced from 4/1 to 1/1, the sensitivity was gradually reduced. The optimal film sensitivity was obtained at the gas flow ratio of 4/1, as shown in FIG. 4.

2. Enzyme Immobilization

The enzyme was immobilized by gel entrapment, comprising the following steps.

(1) 5 mg urate oxidase was added to a 50 mL phosphate buffer solution (20 mM, pH 7.0) to form an enzyme solution.

(2) 25 mg PVA-SbQ polymer was mixed with 100 μL enzyme solution to form an enzyme mixing solution.

(3) 1˜3 μL enzyme mixing solution was titrated on the titanium dioxide film.

(4) The sensing unit was placed in the shade for 20˜30 min to achieve dry and stable condition.

(5) The sensing unit was exposed under long wavelength UV light for about 20 min to photopolymerize the PVA-SbQ polymer.

(6) The sensing unit was washed by deionized water to remove unimmobilized enzyme and PVA-SbQ polymer.

(7) The sensing unit was placed in 4° C.-dry environment for 12 hours to return the stable condition.

(8) The sensing unit was washed by deionized water to remove impurities on the surface of the enzyme sensing film.

(9) The uricase enzyme sensing unit was prepared.

Uric Acid-Containing Test Solution Preparation

A normal person has a uric acid value of about 2˜7 mg/dL. A person with hyperuricemia, however, has a uric acid value exceeding 9 mg/dL. In the example, uric acid-containing test solutions with various concentrations from 4 mg/dL to 10 mg/dL were prepared and the pH value thereof was set to 7 for simulating actual human physiology. The preparation method was mentioned as follows.

(1) 136 g KHPO₄ was added to 50 mL deionized water with stirring to form a slanting acidic KH₂PO₄ buffer solution (20 mM, pH 4.8).

(2) 174 g KHPO₄ was added to 50 mL deionized water with stirring to form a slanting basic KHPO₄ buffer solution (20 mM, pH 8.8).

(3) The KH₂PO₄ buffer solution was titrated to the KHPO₄ buffer solution to form a buffer solution (20 mM, pH 7). The buffer solution was represented as (a) solution.

(4) After stirring the uric acid-containing test solutions, the pH value thereof was measured by a pH meter.

(5) 100 mg uric acid reagent was added to 1000 mL buffer solution to form a test solution having concentration of 10 mg/dL. The uric acid-containing test solution was represented as (b) solution.

(6) 60 mL (a) solution was mixed with 40 mL (b) solution to form a test solution having concentration of 4 mg/dL.

(7) 40 mL (a) solution was mixed with 60 mL (b) solution to form a test solution having concentration of 6 mg/dL.

(8) 20 mL (a) solution was mixed with 80 mL (b) solution to form a test solution having concentration of 8 mg/dL.

Notably, the buffer solution and uric acid-containing test solution must be preserved at 5° C.˜10° C. and avoid high temperature and direct sunlight.

Uric Acid Concentration Measurement

The measurement data was processed by an instrument amplifier LT1167 and a control system HP VEE program connected with a high impedance digital electric meter HP 34401A and a PC. The LT1167 is a front-end detection circuit of the uricase enzyme sensing unit. The uricase enzyme sensing unit was immersed in various test solutions, respectively. After measuring, a response curve of output voltage of the sensing unit was obtained. The measurement method is as follows.

1. To prevent LT1167 from pulse voltage damage at power-on, it must be ensured that the DC current supply is not connected to the LT1167. Simultaneously, the high impedance digital electric meter HP 34401A was warmed up for 5 min to reduce measurement errors.

2. The DC current supply was connected to the LT1167 and the uricase enzyme sensing unit was connected to the input port of the LT1167. The measuring data obtained from the output port thereof was read out by a multi-electric meter. The data was then transmitted to the PC through an interface card. During measurement, data and parameters were measured and controlled by the HP VEE program.

3. The reference electrode and the uricase enzyme sensing unit were immersed in a phosphate buffer solution (PBS) for few seconds to achieve stability. The output voltage was then recorded by the PC.

4. The reference electrode and the uricase enzyme sensing unit were removed to a uric acid-containing test solution.

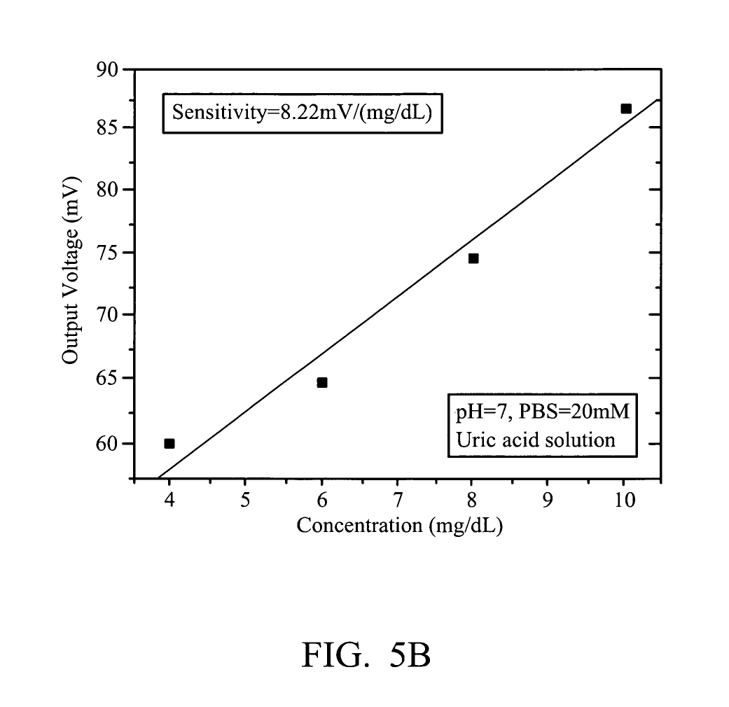

FIG. 5A shows a relationship between response voltage and time (V-T curve) of the uricase enzyme sensing unit immersed in test solutions with various concentrations.

In FIG. 5A, before 25 sec, the uricase enzyme sensing unit was immersed in the phosphate buffer solution for stabilization and provide a base voltage. After 25 sec, the sensing unit was removed to the uric acid-containing test solution. According to response voltages corresponding to various uric acid concentrations, the sensitivity curve of the uricase enzyme sensing unit was obtained, as shown in FIG. 5B. In the example, the uricase enzyme sensing unit has a response time of about 75˜100 sec. Generally, the response time is defined as the time in which the response voltage is increased from zero to 90%.

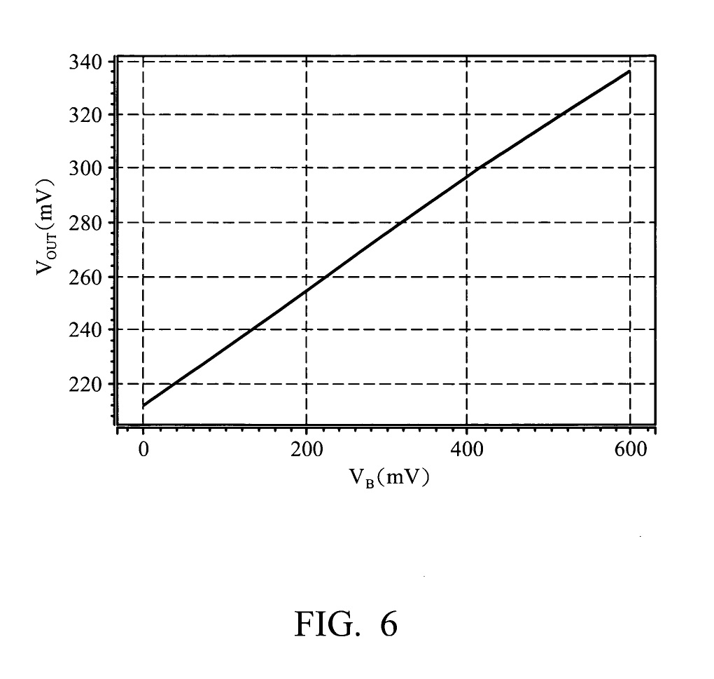

To keep the MOSFET operation in the triode region and ensure that V_{SB} was positive, V_{G} was set to 1V and V_{B} was swept from −1.65V to 0V. The results indicated that the output voltage (Vₒᵤₜ) and the substrate voltage (V_{B}) are proportionate, thus a linear relationship between Vₒᵤₜ and V_{T} was acquired, as shown in FIG. 6. According to the trend of the curve, a correct circuit design can be demonstrated.

While the invention has been described by way of example and in terms of preferred embodiment, it is to be understood that the invention is not limited thereto. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A uricase enzyme biosensor, comprising:
a metal oxide semiconductor field effect transistor;
a sensing unit comprising a substrate, a titanium dioxide film formed thereon and a uricase enzyme sensing film formed on the titanium dioxide film; and
a conductive wire connecting the metal oxide semiconductor field effect transistor and the sensing unit.

2. The uricase enzyme biosensor as claimed in claim 1, wherein the substrate is a semiconductor substrate.

3. The uricase enzyme biosensor as claimed in claim 1, wherein the conductive wire comprises an aluminum wire.

4. The uricase enzyme biosensor as claimed in claim 1, further comprising an insulating layer covering the surface of the sensing unit, exposing the uricase enzyme sensing film.

5. The uricase enzyme biosensor as claimed in claim 4, wherein the insulating layer comprises epoxy.

6. A method of fabricating a uricase enzyme biosensor, comprising:
providing a metal oxide semiconductor field effect transistor;
providing a sensing unit comprising a substrate, a titanium dioxide film formed thereon and a uricase enzyme sensing film formed on the titanium dioxide film; and
providing a conductive wire to connect the metal oxide semiconductor field effect transistor and the sensing unit.

7. The method of fabricating a uricase enzyme biosensor as claimed in claim 6, wherein the substrate is suitable for the deposition of TiO2 film.

8. The method of fabricating a uricase enzyme biosensor as claimed in claim 6, wherein the titanium dioxide film is formed on the substrate by sputtering.

9. The method of fabricating a uricase enzyme biosensor as claimed in claim 8, wherein the sputtering utilizes reaction gases comprising argon and oxygen.

10. The method of fabricating a uricase enzyme biosensor as claimed in claim 9, wherein argon and oxygen have a flow ratio of about 1:1˜4:1.

11. The method of fabricating a uricase enzyme biosensor as claimed in claim 8, wherein the sputtering is radio frequency (RF) sputtering.

12. The method of fabricating a uricase enzyme biosensor as claimed in claim 8, wherein the sputtering has a working pressure of about 10˜40 mTorr, a sputtering duration of about 0.5˜1.5 hour and a RF power of about 120˜180 W.

13. The method of fabricating a uricase enzyme biosensor as claimed in claim 6, wherein the uricase enzyme sensing film is formed on the titanium dioxide film by gel entrapment.

14. The method of fabricating a uricase enzyme biosensor as claimed in claim 13, wherein the steps of the gel entrapment comprise
mixing a light-sensitive polymer and a urate oxidase in a phosphate buffer solution;
titrating the solution on the titanium dioxide film; and
photopolymerizing the solution to form a uricase enzyme sensing film immobilized on the titanium dioxide film.

15. The method of fabricating a uricase enzyme biosensor as claimed in claim 14, wherein the light-sensitive polymer comprises polyvinyl alcohol.

16. The method of fabricating a uricase enzyme biosensor as claimed in claim 14, wherein the light-sensitive polymer and the urate oxidase solution have a weight ratio of about 5:1˜30:1.

17. The method of fabricating a uricase enzyme biosensor as claimed in claim 14, wherein the solution is photopolymerized by exposure of UV light.

18. The method of fabricating a uricase enzyme biosensor as claimed in claim 14, wherein the urate oxidase is entrapped by the light-sensitive polymer to form the uricase enzyme sensing film.

19. The method of fabricating a uricase enzyme biosensor as claimed in claim 6, wherein the conductive wire is an aluminum wire.

20. The method of fabricating a uricase enzyme biosensor as claimed in claim 6, further comprising covering an insulating layer over the surface of the sensing unit, exposing the uricase enzyme sensing film.

21. The method of fabricating a uricase enzyme biosensor as claimed in claim 20, wherein the insulating layer comprises epoxy.

22. A uricase enzyme sensing system, comprising:
a uricase enzyme biosensor as claimed in claim 1;
a reference electrode applying a stabilized voltage;
a semiconductor characteristic instrument disposed on the uricase enzyme biosensor and connected with the reference electrode by a conductive wire; and
a light-isolation container containing the sensing unit of the uricase enzyme biosensor, the reference electrode and a test solution.

23. The uricase enzyme sensing system as claimed in claim 22, wherein the reference electrode is an Ag/AgCl reference electrode.

24. The uricase enzyme sensing system as claimed in claim 22, wherein the semiconductor characteristic instrument is a current/voltage instrument.

25. The uricase enzyme sensing system as claimed in claim 24, wherein the semiconductor characteristic instrument measures drain current and gate voltage.

26. The uricase enzyme sensing system as claimed in claim 22, wherein the conductive wire is an aluminum wire.

27. The uricase enzyme sensing system as claimed in claim 22, wherein the test solution is a uric acid-containing solution.

28. A sensing circuit, comprising:
a uricase enzyme biosensor as claimed in claim 1;
a first operational amplifier comprising an output port, a negative-phase input port and a non-negative-phase input port, wherein the output port and the negative-phase input port are connected to the uricase enzyme biosensor, and the non-negative-phase input port is connected to a first current source and a first port of a resistance; and
a second operational amplifier comprising an output port, a negative-phase input port and a non-negative-phase input port, wherein the output port and the negative-phase input port are connected to a second port of the resistance, and the non-negative-phase input port is connected to a second current source and the uricase enzyme biosensor.

29. The sensing circuit as claimed in claim 28, wherein the first and second operational amplifiers are negative feedback voltage buffers.

30. The sensing circuit as claimed in claim 28, wherein the sensing circuit exhibits two-stage operational amplification.
